(19) United States
(12) Patent Application Publication
(10) Pub. No.: US 2026/0362016 A1
(43) Pub. Date: Nov. 12, 2026

(54) DYNAMIC LUMINANCE CALIBRATION (D.L.C.) METHOD

(71) Applicant: Weyo Surgical Technology Ltd., Jiangsu (CN)

(72) Inventors: Zhiqiang Ren, Jiangsu (CN); Peng Zou, Jiangsu (CN); Mingjian Sun, Jiangsu (CN)

(73) Assignee: Weyo Surgical Technology Ltd., Jiangsu (CN)

(21) Appl. No.: 19/485,275

(22) PCT Filed: Mar. 19, 2025
(86) PCT No.: PCT/CN2025/083297

§ 371 Date: Nov. 17, 2025

(30) Foreign Application Priority Data
Jun. 13, 2024 (CN) 202410756959.1

(51) Int. Cl. H04N 23/76; H04N 23/50; H04N 23/71; H04N 23/73

(52) U.S. Cl. CPC H04N 23/76; H04N 23/555; H04N 23/71; H04N 23/73

(57) ABSTRACT

The present invention relates to the technical field of endoscope imaging, in particular to a dynamic luminance calibration (D.L.C.) method. The method includes the following steps: performing auto metering on a dynamic video; performing auto dimming on the dynamic video subjected to metering, wherein the auto metering and the auto dimming fall within a category of auto exposure; and performing dynamic luminance adjustment on the video subjected to dimming, so that the picture luminance of the video is always in a reasonable state. The present invention, by designing a dynamic luminance calibration (D.L.C.) method, enables rapid and effective adjustment of exposure and picture luminance. During exposure adjustment, the image luminance rapidly converges to the vicinity of the target luminance, the convergence process is smooth with comfortable visual perception, and the luminance remains stable after the completion of convergence without breathing effect.

### TECHNICAL FIELD

The present invention relates to the technical field of endoscope imaging, and in particular to a dynamic luminance calibration (D.L.C.) method.

### RELATED ART

In the field of endoscopic imaging, very high requirements are placed on the luminance stability of pictures. On the one hand, a stable picture can reduce the doctor's visual fatigue, and on the other hand, a stable picture can allow the doctor to face the surgical scene more calmly.

For image sensors, image luminance (LUMA) is determined by the exposure time (ET) and the analog gain amplification factor (GAIN). The working principle of the image sensor determines that the exposure time (ET) and the analog gain amplification factor (GAIN) are basically linearly related to the image luminance, so the image luminance can be expressed as

LUMA=k*GAIN*ET.

Therefore, the auto exposure algorithm needs to obtain the luminance of the current frame image and calculate the exposure time and analog gain magnification factor required for the next frame.

Due to the existence of narrow and long channels in medical endoscope application scenarios, dark regions may still exist in pictures upon adjustment via the auto exposure algorithm, resulting in suboptimal luminance representation in dark regions of the pictures, thereby causing several typical issues as follows:

(1) The dimming is unstable, which causes non-linear luminance changes between frames and picture flickering during dimming.

(2) The dimming speed is slow, which results in long-term overexposure or underexposure of the pictures.

(3) Since the actual exposure register values are discrete, the dimming amplitude may be inaccurate, which results in that the picture luminance is unstable during the dimming, the auto exposure fails to reach optimal luminance, causing intermittent luminance fluctuations.

(4) Overly bright proximal views and excessively dark distal views are presented in the narrow and long lumens.

### SUMMARY OF INVENTION

In view of this, an objective of the present invention is to propose a dynamic luminance calibration (D.L.C.) method to solve the existing problem that due to the existence of narrow and long channels in medical endoscope application scenarios, dark regions may still exist in pictures upon adjustment via the auto exposure algorithm, resulting in suboptimal luminance representation in dark regions of the pictures.

Based on the above objective, the present invention provides a dynamic luminance calibration (D.L.C.) method, comprising the following steps:
S1: performing auto metering on a dynamic video;
S2: performing auto dimming on the dynamic video subjected to metering, wherein the auto metering and the auto dimming fall within a category of auto exposure; and
S3: performing dynamic luminance adjustment on the video subjected to dimming, so that the picture luminance of the video is always in a reasonable state;
Step S1 comprises image valid region detection and auto metering, wherein the image valid region detection specifically comprises the following substeps:
S1.1: selecting valid pixels in an image using a threshold parameter TH_valid; and
S1.2: for each pixel (x,y) in an image I, if I_{c}(x,y)>TH_valid, considering that the pixel (x,y) is a valid pixel, wherein c represents RGB triple channels, and a ratio of a number of valid pixels to a total number of pixels is denoted as ratio, wherein a TH_valid value is set to 8 by default;
and the auto metering specifically comprises the following substeps:
S1.3: dividing the image into a plurality of blocks, with a total number of the blocks denoted as total_block, and calculating an average luminance Lᵢ of each block i sized as s*s, wherein a formula of the average luminance Lᵢ is as follows:

$${L}_{i} ( x , y ) = ⌊ 0.2126 * {R}_{i} ( x , y ) + 0.7152 * {G}_{i} ( x , y ) + 0.0722 * {B}_{i} ( x , y ) ⌋ ,$$

$${L}_{i} = \frac{1}{{s}^{2}} {\sum }_{x = 1}^{s} {\sum }_{y = 1}^{s} {L}_{i} ( x , y ) ,$$

wherein, Lᵢ(x,y) indicates a luminance of a pixel in the block, Rᵢ(x,y), Gᵢ(x,y), Bᵢ(x,y) indicates values of the pixel (x,y) in the RGB triple channels, respectively, and └ ┘ indicates a floor operation;
S1.4: calculating a number of selected blocks;

block=ratio*total_block; and
S1.5: sorting the average luminances of all blocks in descending order, so that a metering result is;

$${Y}_{L} = ⌊ \frac{S}{block} {\sum }_{j = 1}^{block} {L}_{j} ⌋ + ⌊ \frac{1 - S}{{total}_{b l o c k} - block - 1} {\sum }_{block + 1}^{total \_ block} {L}_{j} ⌋ ,$$

wherein Lⱼ indicates the average luminance of the selected j-th block, and S is a tolerance parameter which can be set to any value between 0 and 1.

Preferably, for an N-th frame image in a video stream, input conditions for an auto dimming module are: a current image luminance Y_{N}, a previous-frame image luminance Y_{N-1}, a target image luminance TY, exposure times ET_{N} and ET_{N-1}, analog gain amplification factors GAIN_{N} and GAIN_{N-1}; and a process of determining whether a dimming is required in S2 is as follows:
S2.1: when abs(TY−Y_{N})<range, i.e., a current picture luminance is slightly different from a target luminance, maintaining exposure parameters unchanged, and exiting the auto dimming, wherein abs( ) represents an absolute value function, and range represents a threshold constant; and
S2.2: when abs(TY−Y_{N})>range, entering a dimming process.

Preferably, the dimming process in step S2.2 comprises correcting an exposure model and calculating the exposure parameters, and the correcting the exposure model specifically comprises the following substeps:
S2.21: according to a working principle of an image sensor, under the condition of unchanged scenes, providing the following relationship:

GAIN_{N+1}−GAIN_{N}=K₁*(Y_{N+1}−Y_{N}),

ET_{N+1}−ET_{N}=K₂*(Y_{N+1}−Y_{N});
S2.22: setting K to an initial value which needs to be corrected in time for changing scenes, and in the dimming process, when one of GAIN and ET changes, recalculating a value of K of a current frame based on a variation of the luminance, and when both change at the same time, expressing the value of K as follows:

$${K}_{1} = {K}_{1} * \sqrt[2]{\frac{{( {Y}_{N + 1} - {Y}_{N} )}^{2}}{( {GAIN}_{N + 1} - {GAIN}_{N} ) * ( E {T}_{N + 1} - E {T}_{N} )}} ,$$

$${K}_{2} = {K}_{2} * \sqrt[2]{\frac{{( {Y}_{N + 1} - {Y}_{N} )}^{2}}{( {GAIN}_{N + 1} - {GAIN}_{N} ) * ( E {T}_{N + 1} - E {T}_{N} )} ;}$$

S2.23: in order to improve the robustness of auto exposure during endoscope movement, taking the value of K as a weighted value of the current frame and the previous frames, and after an estimated value of K is obtained, estimating a variation of the exposure parameters based on a difference between the current frame luminance and the target luminance.

Preferably, the calculating the exposure parameters specifically comprises the following substeps:
S2.24: introducing n damping coefficients p to further adjust an intermediate process of dimming while a dimming speed can also be adjusted through the damping coefficients, wherein the damping coefficients p are expressed as follows:

$$p = \{ \begin{matrix}{p}_{1} , & th \leq {th}_{1} \\ ⋮ & ⋮ \\ {p}_{i} , & {th}_{i - 1} < th \leq {th}_{i} \\ ⋮ & ⋮ \\ {p}_{n} , & {th}_{n - 1} < th \leq {th}_{n}\end{matrix} ,$$

$$th = ⌊ \frac{abs ( TY - {Y}_{N} )}{{Y}_{N}} ⌋ ;$$

S2.25: since increasing the exposure time helps improve the signal-to-noise ratio, giving priority to using the exposure time during dimming, and adjusting the analog gain amplification factor only when the exposure time is insufficient to meet the luminance requirement, when GAIN=1, disenabling GAIN, and adjusting ET:

ΔET=└p*K₂*(TY−Y_{N})┘,

ET_{N+1}=ET_{N}+ΔET,

calculating and issuing an actual register value Reg_{ET} to complete the dimming of the current frame.

Preferably, when GAIN≠1, GAIN is adjusted:

ΔGAIN=└p*K₁*(TY−Y_{N})┘,

GAIN_{N+1}=GAIN_{N}+ΔGAIN,
the actual register value Reg_{GAIN} is calculated at this time, and when ΔGAIN is less than a threshold th_g, it is further corrected through D_{gain};
a difference between a magnification factor corresponding to the actual register value and an actual magnification factor of the previous frame is denoted as A, then:

A*abs(D_{gain}−1)=p*K₁*(TY−Y_{N}),
wherein D_{gain}>0, the adjusted D_{gain} can be calculated based on the relational expression, and finally, Reg_{ET} and Reg_{GAIN} are calculated for ending the dimming, and an N+1 frame image is obtained by multiplying by D_{gain} on the N+1 frame image.

Preferably, in step S3, the following substeps are included:
S3.1: dynamically determining whether to adjust a picture;
S3.2: performing adaptive dark region brightening; and
S3.3: performing dynamical contrast enhancement.

Preferably, the dynamically determining whether to adjust a picture in step S3.1 specifically comprises the following substeps:
S3.11: first, converting the image from an RGB domain to a YUV domain using a formula as follows:

Y=0.1826*R+0.6142*G+0.062*B+16,

U=−0.1006*R−0.3386*G+0.4392*B+128,

V=0.4392*B−0.3989*G−0.0403*B+128; and
S3.12: performing histogram statistics on the Y channel, wherein during the histogram statistics, the idea of “image valid region detection in step S1” continues to be used, that is, only pixels with a luminance greater than TH_valid are counted; after a luminance histogram Hist_L is obtained, the numbers of pixels across all grayscale levels in Hist_L are summed to obtain Hist_L_sum, then the number of pixels across each grayscale level in Hist_L is divided by Hist_L_sum to obtain a weight for each grayscale level, as expressed in the following formula:

Hist_L_wⱼ=Hist_Lⱼ/Hist_L_sum;
S3.13: performing weighted summation on the weight and each grayscale value to obtain a weighted grayscale value of the image, as expressed in the following formula:

$$gray\_avg = {\sum }_{j = 0}^{255} Hist\_L {\_w}_{j} * j ,$$

in the histogram Hist_L, the number of pixels with grayscale levels less than TH_GT is counted as left_num and the number of pixels with grayscale levels greater than or equal to TH_GT is counted as right_num, wherein TH_GT defaults to 90, and r_l_ratio is a ratio of the number of pixels on the right side of TH_GT to the number of pixels on the left side of TH_GT, as expressed in the following formula:

r_l_ratio=right_num/left_num; and
S3.14: when gray_avg is greater than or equal to TH_GT, considering that the picture is normal and requires no changes;
when r_l_ratio is greater than or equal to 1, considering that the picture is normal and requires no changes; except for the above two cases, considering that the picture is generally dark and requires dynamic brightening.

Preferably, the adaptive dark region brightening in step S3.2 specifically comprises the following substeps:
S3.21: when a value of gray_avg is small, considering that the picture has a low overall luminance and requires more enhancement for a dark region, in which case a bimodal distribution may appear such that it is necessary to introduce a r_l_ratio ratio, and the closer the ratio approaches 1, the higher the overall differentiation of the picture, indicating that the luminance/darkness distribution is appropriate, and there is no need to brighten the dark region.

S3.22: designing a curve dynamically adjusted based on gray_avg and r_l_ratio with a formula as follows:

f(x)=(x^{b}*c^{d})/(x^{b}*c^{d}+(1−x)^{d}*a^{b}),
wherein x is a grayscale value ranging from 0 to 255, f(x) indicates that an original grayscale value will be mapped to a new grayscale value, wherein a is 0.3, and d is 1;
c is dynamically adjusted based on gray_avg, with a formula as follows:

c_i=min((TH_GT−gray_avg)/(TH_GT−40),1),

c=0.3+c_i*0.3;
b is dynamically adjusted based on r_l_ratio and gray_avg, wherein min represents a smaller one of the two values, with a formula as follows:

$$new\_r \_l \_ratio = \frac{( r\_l {\_ratio}^{3} * 0.3 {5}^{3} )}{( r\_l {\_ratio}^{3} * 0.3 {5}^{3} + {( 1 - r\_l \_ratio )}^{3} * 0 . {5}^{3} )} ,$$

$$b\_i = 1 - c\_i * ( 1 - new\_r \_l \_ratio ) ,$$

$$b = min ( 0.35 + 0.65 * b\_i , 1 ) ;$$

and
S3.23: applying a grayscale value of a new f(x) to the Y channel of the image in the YUV domain to obtain an image YUV_new which is brightened newly.

Preferably, the dynamic contrast enhancement in step S3.3 comprises the following substeps:
S3.31: first, converting the image YUV_new to an image RGB_new, i.e., converting the image from the YUV domain to the RGB domain;
S3.32: enhancing the RGB_new using an adaptive linear contrast method, with a specific enhancement formula as follows:

$${res \_ p}_{m}^{n} = clip ( ( {p}_{m}^{n} - TH\_GT ) * c\_f + TH\_GT , 0 , 255 ) ,$$

wherein, m in

$${p}_{m}^{n}$$

represents a pixel at a certain position, and n represents a certain color channel in RGB, c_f is a contrast enhancement factor, and a c_f formula is as follows:

c_f=1.4−0.4*b_i,
wherein, a clip operation means that when a calculation result is less than 0, it will be truncated to 0, and when it is greater than 255, it will be truncated to 255, wherein

$${res \_ p}_{m}^{n}$$

is a final graphical result.

Preferably, in step S3.31, a formula for converting the image from the YUV domain to the RGB domain is as follows:

R=1.1644*(Y−16)+1.7928*(V−128),

G=1.1644*(Y−16)−0.2133*(U−127)−0.533*(V−128),

B=1.1644*(Y−16)+2.1124*(U−128).

The present invention has the following advantageous effects:

I. The valid region detection is added, which improves the adaptability of auto exposure in different endoscopy scenarios, and makes the auto metering results more stable.

II. The dimming process can correct the exposure theoretical model in real time, thereby ensuring the accuracy of the dimming model and improving the robustness of auto exposure during endoscope movement.

III. The damping coefficient is introduced into the dimming process to ensure the stability and speed of dimming. Since increasing the exposure time helps to improve the signal-to-noise ratio, the exposure time should be used first in the dimming process, and the analog gain amplification factor is adjusted when the exposure time is insufficient to meet the luminance requirements, thereby improving the accuracy of the dimming.

IV. For the scene of narrow and long lumens in endoscopic images, a dynamic luminance adjustment algorithm is designed, so that when the picture is dark, the luminance of the dark region of the picture can be adaptively adjusted, and when the luminance is dynamically adjusted, the adaptive contrast method is used to ensure that the picture still maintains a high contrast.

V. In summary, this method enables rapid and effective adjustment of exposure and picture luminance. During exposure adjustment, the image luminance quickly converges to the vicinity of the target luminance, the convergence process is smooth with comfortable visual perception, and the luminance remains stable after the completion of convergence without breathing effect. When the picture is dark, the luminance can be dynamically enhanced, and during dynamic luminance adjustment, the picture shall maintain sufficient contrast, so that the picture luminance is always in a reasonable state.

### BRIEF DESCRIPTION OF DRAWINGS

To describe the technical solutions in the embodiments of the present invention or in the prior art more clearly, the accompanying drawings required for describing the embodiments or the prior art are briefly introduced below. It is obvious that the accompanying drawings in the following description only show embodiments of the present invention, and other drawings may be obtained based on these drawings by those of ordinary skill in the art without creative efforts.

FIG. 1 is an overall flowchart according to an embodiment of the present invention;

FIG. 2 is an auto exposure flowchart according to an embodiment of the present invention;

FIG. 3 is an auto dimming flowchart according to an embodiment of the present invention;

FIG. 4 is a dynamic luminance adjustment flowchart according to an embodiment of the present invention;

FIG. 5 is an original image without dynamic luminance adjustment according to an embodiment of the present invention;

FIG. 6 is a result diagram illustrating the application of adaptive dark region brightening according to an embodiment of the present invention; and

FIG. 7 is a result diagram illustrating the combined application of adaptive dark region brightening and dynamic contrast enhancement according to an embodiment of the present invention.

### DESCRIPTION OF EMBODIMENTS

To make the objectives, technical solutions and advantages of the present invention clearer, the present invention is further described in detail below with reference to specific embodiments and accompanying drawings.

It should be noted that, unless defined otherwise, technical or scientific terms used in the embodiments of the present invention shall have the same meanings as commonly understood by one of ordinary skill in the art to which the present invention belongs. The terms “first”, “second”, and similar expressions used in the present invention do not denote any order, quantity, or importance, but rather are used to distinguish different components. The terms “comprise”, “include” or similar expressions mean that the elements or items preceding the terms cover the elements or items listed after the terms and their equivalents, without excluding other elements or items. The terms “connect”, “couple” or similar expressions are not limited to physical or mechanical connections, but may include electrical connections, whether direct or indirect. The terms “upper”, “lower”, “left”, “right” or similar expressions are only used to indicate relative positional relationships. When the absolute positions of described objects change, the relative positional relationships may change accordingly.

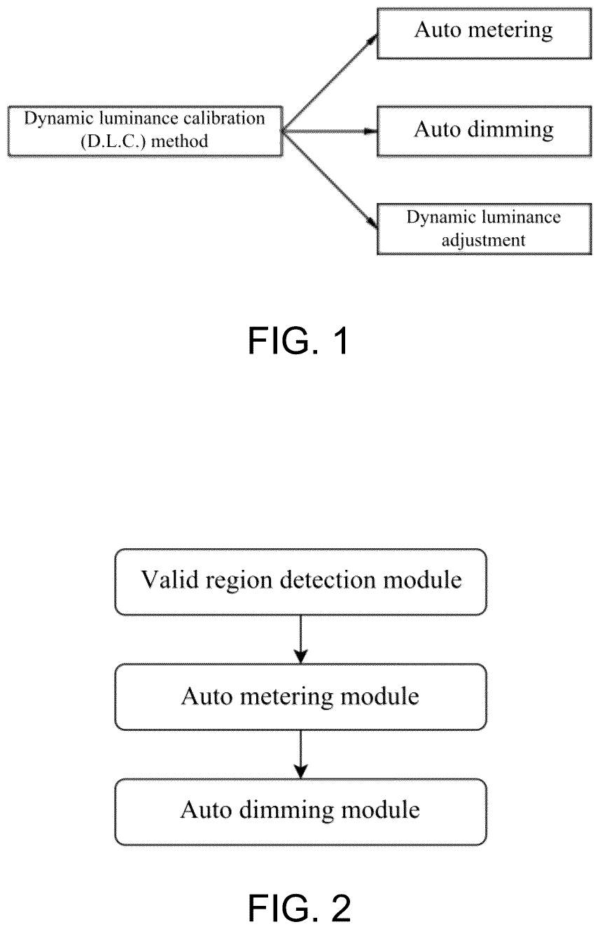

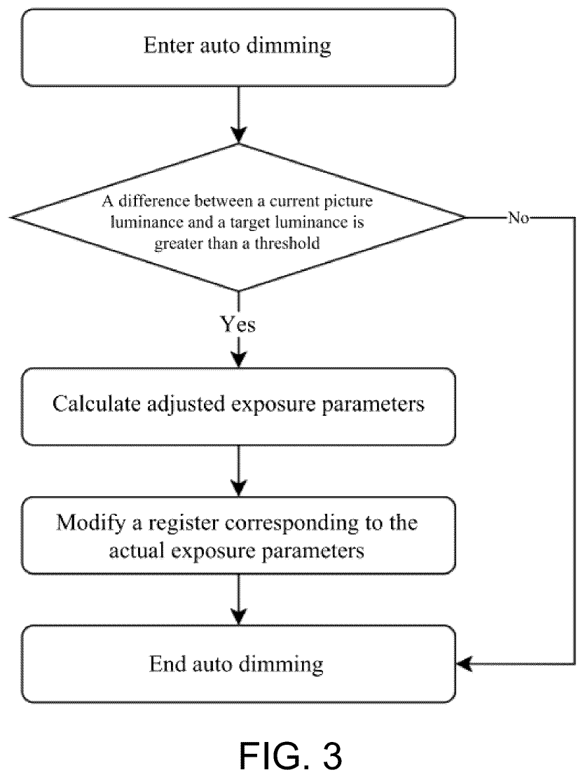

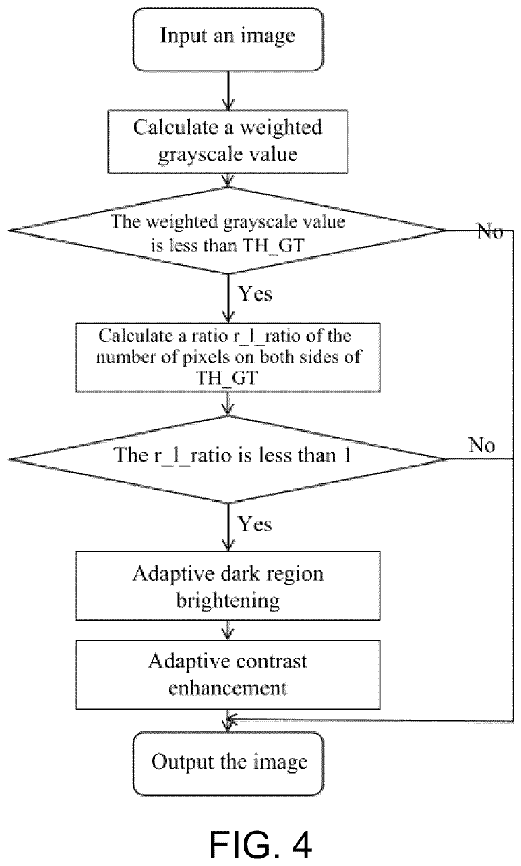

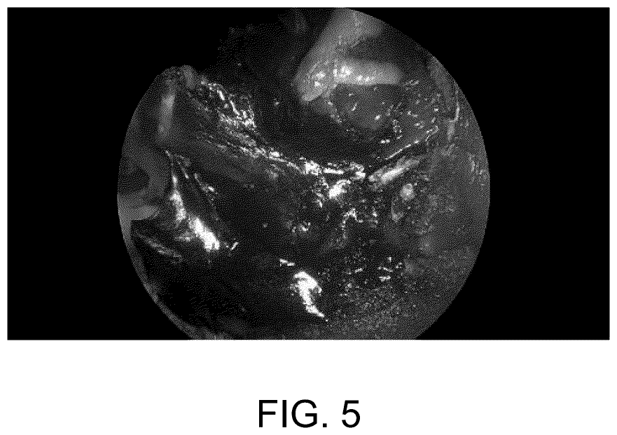

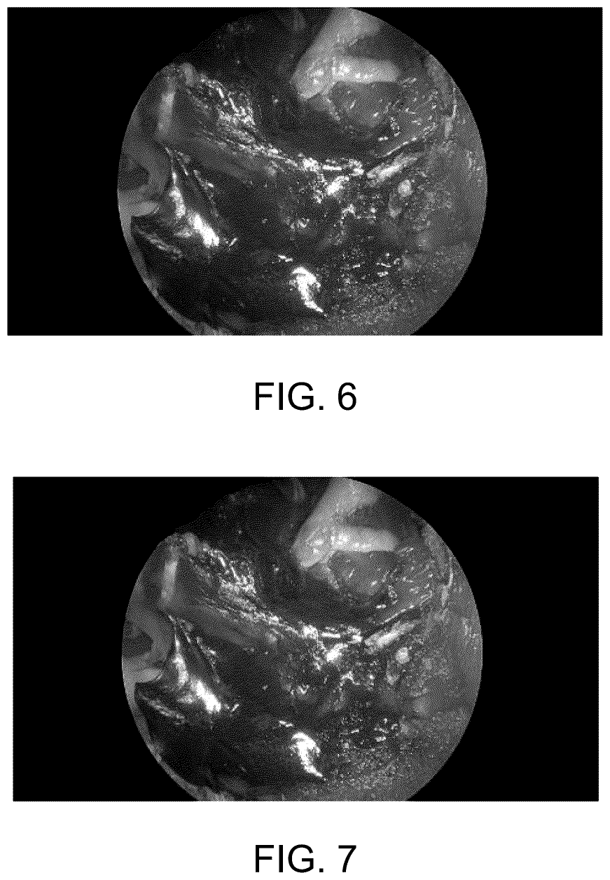

As shown in FIG. 1, FIG. 2, FIG. 3, FIG. 4, FIG. 5, FIG. 6 and FIG. 7, a dynamic luminance calibration (D.L.C.) method includes the following steps:

S1: Auto metering is performed on a dynamic video.

Step S1 includes image valid region detection and auto metering, wherein the image valid region detection specifically includes the following substeps:

S1.1: Valid pixels in an image are selected using a threshold parameter TH_valid.

S1.2: For each pixel (x,y) in an image I, if I_{c}(x,y)>TH_valid, it is considered that the pixel (x,y) is a valid pixel, where c represents RGB triple channels, and a ratio of a number of valid pixels to a total number of pixels is denoted as ratio, where a TH_valid value is set to 8 by default.

The auto metering specifically includes the following substeps:

S1.3: The image is divided into a plurality of blocks, where the size of each block may be 8*8, 16*16 or 32*32 and a total number of the blocks is denoted as total_block; and an average luminance Lᵢ of each block i sized as s*s is calculated, where a formula of the average luminance Lᵢ is as follows:

$${L}_{i} ( x , y ) = ⌊ 0.2126 * {R}_{i} ( x , y ) + 0.7152 * {G}_{i} ( x , y ) + 0.0722 * {B}_{i} ( x , y ) ⌋ ,$$

$${L}_{i} = \frac{1}{{s}^{2}} {\sum }_{x = 1}^{s} {\sum }_{y = 1}^{s} {L}_{i} ( x , y ) ,$$

where, Lᵢ(x,y) indicates a luminance of a pixel in the block, Rᵢ(x,y), Gᵢ(x,y), Bᵢ(x,y) indicates values of the pixel (x,y) in the RGB triple channels, respectively, and └ ┘ indicates a floor operation; and this calculation method has been proven to be highly robust for bleeding scenarios.

S1.4: A number of selected blocks is calculated;

block=ratio*total_block.

S1.5: The average luminances of all blocks are sorted in descending order, so that a metering result is;

$${Y}_{L} = ⌊ \frac{S}{block} {\sum }_{j = 1}^{block} {L}_{j} ⌋ + ⌊ \frac{1 - S}{{total}_{block} - block - 1} {\sum }_{block + 1}^{total \_ block} {L}_{j} ⌋ ,$$

where Lⱼ indicates the average luminance of the selected j-th block, and S is a tolerance parameter which can be set to any value between 0 and 1.

S2: Auto dimming is performed on the dynamic video subjected to metering, where the auto metering and the auto dimming fall within a category of auto exposure.

For an N-th frame image in a video stream, input conditions for an auto dimming module are: a current image luminance Y_{N}, a previous-frame image luminance Y_{N-1}, a target image luminance TY, exposure times ET_{N} and ET_{N-1}, analog gain amplification factors GAIN_{N} and GAIN_{N-1}; and a process of determining whether a dimming is required in S2 is as follows:

S2.1: When abs(TY−Y_{N})<range, i.e., a current picture luminance is slightly different from a target luminance, exposure parameters are maintained unchanged, and the auto dimming is exited, wherein abs( ) represents an absolute value function, and range represents a threshold constant.

S2.2: When abs(TY−Y_{N})>range, a dimming process enters.

The dimming process in step S2.2 includes correcting an exposure model and calculating the exposure parameters, and the correcting the exposure model specifically includes the following substeps:

S2.21: According to a working principle of an image sensor, under the condition of unchanged scenes, the following relationship is provided:

GAIN_{N+1}−GAIN_{N}=K₁*(Y_{N+1}−Y_{N}),

ET_{N+1}−ET_{N}=K₂*(Y_{N+1}−Y_{N}).

S2.22: K is set to an initial value which needs to be corrected in time for changing scenes, and in the dimming process, when one of GAIN and ET changes, a value of K of a current frame is recalculated based on a variation of the luminance, and when both change at the same time, the value of K is expressed as follows:

$${K}_{1} = {K}_{1} ⋆ \sqrt[2]{\frac{{( {Y}_{N + 1} - {Y}_{N} )}^{2}}{( G A I {N}_{N + 1} - G A I {N}_{N} ) * ( E {T}_{N + 1} - E {T}_{N} )}} ,$$

$${K}_{2} = {K}_{2} * \sqrt[2]{\frac{{( {Y}_{N + 1} - {Y}_{N} )}^{2}}{( G A I {N}_{N + 1} - G A I {N}_{N} ) * ( E {T}_{N + 1} - E {T}_{N} )}} .$$

S2.23: In order to improve the robustness of auto exposure during endoscope movement, the value of K may be a weighted value of the current frame and the previous frames, and after an estimated value of K is obtained, a variation of the exposure parameters may be estimated based on a difference between the current frame luminance and the target luminance.

The calculating the exposure parameters specifically includes the following substeps:

Some thresholds should be set in the dimming process to limit the adjustment range of exposure parameters to avoid excessive or insufficient adjustments. For example, the maximum ΔET/ET_{N} and the maximum ΔGAIN/GAIN_{N} are set.

S2.24: n damping coefficients p are introduced to further adjust an intermediate process of dimming while a dimming speed can also be adjusted through the damping coefficients, where the damping coefficients p are expressed as follows:

$$p = \{ \begin{matrix}{p}_{1} , & t h \leq t {h}_{1} \\ ⋮ & ⋮ \\ {p}_{i} , & t {h}_{i − 1} < t h \leq t {h}_{i} \\ ⋮ & ⋮ \\ {p}_{n} , & t {h}_{n − 1} < t h \leq t {h}_{n}\end{matrix} ,$$

$$th = ⌊ \frac{abs ( TY - {Y}_{N} )}{{Y}_{N}} ⌋ .$$

S2.25: Since increasing the exposure time helps improve the signal-to-noise ratio, the exposure time should be used first during dimming, and the analog gain amplification factor is adjusted again when the exposure time is insufficient to meet the luminance requirement, when GAIN=1, GAIN is disenabled, and ET is adjusted:

ΔET=└p*K₂*(TY−Y_{N})┘,

ET_{N+1}=ET_{N}+ΔET.

An actual register value Reg_{ET} is calculated and issued to complete the dimming of the current frame.

When GAIN≠1, GAIN is adjusted:

ΔGAIN=└p*K₁*(TY−Y_{N})┘,

GAIN_{N+1}=GAIN_{N}+ΔGAIN.

The actual register value Reg_{GAIN} is calculated at this time, and when ΔGAIN is less than a threshold th_g, it is further corrected through D_{gain}.

A difference between a magnification factor corresponding to the actual register value and an actual magnification factor of the previous frame is denoted as A, then:

A*abs(D_{gain}−1)=p*K₁*(TY−Y_{N}),
where D_{gain}>0, the adjusted D_{gain} can be calculated based on the relational expression, and finally, Reg_{ET} and Reg_{GAIN} are calculated for ending the dimming, and an N+1 frame image is obtained by multiplying by D_{gain} on the N+1 frame image.

S3: Dynamic luminance adjustment is performed on the video subjected to dimming, so that the picture luminance of the video is always in a reasonable state.

In step S3, the following substeps are included:

S3.1: Whether to adjust a picture is dynamically determined.

The dynamically determining whether to adjust a picture in step S3.1 specifically includes the following substeps:

S3.11: First, the image is converted from an RGB domain to a YUV domain using a formula as follows:

Y=0.1826*R+0.6142*G+0.062*B+16,

U=−0.1006*R−0.3386*G+0.4392*B+128,

V=0.4392*B−0.3989*G−0.0403*B+128.

S3.12: Histogram statistics is performed on the Y channel, where during the histogram statistics, the idea of “image valid region detection in step S1” continues to be used, that is, only pixels with a luminance greater than TH_valid are counted; after a luminance histogram Hist_L is obtained, the numbers of pixels across all grayscale levels in Hist_L are summed to obtain Hist_L_sum, then the number of pixels across each grayscale level in Hist_L is divided by Hist_L_sum to obtain a weight for each grayscale level, as expressed in the following formula:

Hist_L_wⱼ=Hist_Lⱼ/Hist_L_sum.

S3.13: Weighted summation is performed on the weight and each grayscale value to obtain a weighted grayscale value of the image, as expressed in the following formula:

$$gray\_avg = {\sum }_{j = 0}^{255} Hist\_L {\_w}_{j} * j ,$$

In the histogram Hist_L, the number of pixels with grayscale levels less than TH_GT is counted as left_num and the number of pixels with grayscale levels greater than or equal to TH_GT is counted as right_num, where TH_GT defaults to 90, and r_l_ratio is a ratio of the number of pixels on the right side of TH_GT to the number of pixels on the left side of TH_GT, as expressed in the following formula:

r_l_ratio=right_num/left_num.

S3.14: When gray_avg is greater than or equal to TH_GT, it is considered that the picture is normal and requires no changes; and

when r_l_ratio is greater than or equal to 1, it is considered that the picture is normal and requires no changes; except for the above two cases, it is considered that that the picture is generally dark and requires dynamic brightening.

S3.2: Adaptive dark region brightening is performed.

The adaptive dark region brightening in step S3.2 specifically includes the following substeps:

S3.21: When a value gray_avg is small, it is considered that the picture has a low overall luminance and requires more enhancement for a dark region, in which case a bimodal distribution may appear such that it is necessary to introduce a r_l_ratio ratio, and the closer the ratio approaches 1, the higher the overall differentiation of the picture, indicating that the luminance/darkness distribution is appropriate, and there is no need to brighten the dark region.

S3.22: A curve dynamically adjusted based on gray_avg and r_l_ratio is designed, with a formula as follows:

f(x)=(x^{b}*c^{d})/(x^{b}*c^{d}+(1−x)d*a^{b}),
where x is a grayscale value ranging from 0 to 255, f(x) indicates that an original grayscale value will be mapped to a new grayscale value, where a is 0.3, and d is 1.

c is dynamically adjusted based on gray_avg, with a formula as follows:

c_i=min((TH_GT−gray_avg)/(TH_GT−40),1),

c=0.3+c_i*0.3.

b is dynamically adjusted based on r_l_ratio and gray_avg, wherein min represents a smaller one of the two values, with a formula as follows:

$$new\_r \_l \_ratio = \frac{( r\_l {\_ratio}^{3} * 0.3 {5}^{3} )}{( r\_l {\_ratio}^{3} * 0.3 {5}^{3} + {( 1 - r\_l \_ratio )}^{3} * 0 . {5}^{3} )} ,$$

$$b\_i = 1 - c\_i * ( 1 - new\_r \_l \_ratio ) .$$

$$b = min ( 0.35 + 0.65 * b\_i , 1 ) .$$

S3.23: A grayscale value of a new f(x) is applied to the Y channel of the image in the YUV domain to obtain an image YUV_new which is brightened newly.

S3.3: Dynamical contrast enhancement is performed.

When the enhancement curve is applied for certain enhancement, the picture contrast is generally reduced. At this time, a contrast enhancement method is required to restore partial contrast, thereby improving the visual perception of the picture.

The dynamic contrast enhancement in step S3.3 specifically includes the following substeps:

S3.31: First, the image YUV_new to an image RGB_new, i.e., the image is converted from the YUV domain to the RGB domain.

In step S3.31, a formula for converting the image from the YUV domain to the RGB domain is as follows:

R=1.1644*(Y−16)+1.7928*(V−128),

G=1.1644*(Y−16)−0.2133*(U−127)−0.533*(V−128),

B=1.1644*(Y−16)+2.1124*(U−128).

S3.32: The RGB_new is enhanced using an adaptive linear contrast method, with a specific enhancement formula as follows:

$${res\_p}_{m}^{n} = clip ( ( {p}_{m}^{n} - TH\_GT ) * c\_f + TH\_GT , 0 , 255 ) ,$$

where, m in

$${p}_{m}^{n}$$

represents a pixel at a certain position, and n represents a certain color channel in RGB, c_f is a contrast enhancement factor, and a c_f formula is as follows:

c_f=1.4−0.4*b_i,
where, a clip operation means that when a calculation result is less than 0, it will be truncated to 0, and when it is greater than 255, it will be truncated to 255, wherein

$${res\_p}_{m}^{n}$$

is a final graphical result.

Those skilled in the art should understand that the discussion of any of the above embodiments is merely illustrative and is not intended to imply that the scope of the present invention is limited to these examples. Under the concept of the present invention, the technical features in the above embodiments or different embodiments may be combined, the steps may be implemented in any order, and there are many other variations of the different aspects of the present invention as described above, which are not provided in detail for the sake of simplicity.

The embodiments of the present invention are intended to cover all such substitutions, modifications and variations that fall within the broad scope of the appended claims. Therefore, any omission, modification, equivalent substitution, improvement, etc. made within the spirit and principles of the present invention shall be included within the scope of protection of the present invention.

1. A dynamic luminance calibration (D.L.C.) method, characterized by comprising following steps:
S1: performing auto metering on a dynamic video;
S2: performing auto dimming on the dynamic video subjected to metering, wherein the auto metering and the auto dimming fall within a category of auto exposure; and
S3: performing dynamic luminance adjustment on the dynamic video subjected to dimming, so that picture luminance of the dynamic video is always in a reasonable state;
the step S1 comprises image valid region detection and the auto metering, wherein the image valid region detection specifically comprises following substeps:
S1.1: selecting valid pixels in an image using a threshold parameter TH_valid; and
S1.2: for each pixel (x,y) in the image I, if I_{c} (x,y)>TH_valid, considering that the pixel (x,y) is a valid pixel, wherein c represents RGB triple channels, and a ratio of a number of valid pixels to a total number of pixels is denoted as ratio, wherein a value of TH_valid is set to 8 by default;
and the auto metering specifically comprises following substeps:
S1.3: dividing the image into a plurality of blocks, with a total number of the blocks denoted as total_block, and calculating an average luminance Lᵢ of each of the block i sized as s*s, wherein a formula of the average luminance Lᵢ is as follows:
$${L}_{i} ( x , y ) = ⌊ 0.2126 * {R}_{i} ( x , y ) + 0.7152 * {G}_{i} ( x , y ) + 0.0722 * {B}_{i} ( x , y ) ⌋ ,$$
$${L}_{i} = \frac{1}{{s}^{2}} {\sum }_{x = 1}^{s} {\sum }_{y = 1}^{s} {L}_{i} ( x , y ) ,$$
wherein, Lᵢ(x,y) indicates a luminance of a pixel in each of the block, Rᵢ(x,y), Gᵢ(x,y), Bᵢ(x,y) indicates values of the pixel (x,y) in the RGB triple channels, respectively, and └ ┘ indicates a floor operation;
S1.4: calculating a number of selected blocks;
block=ratio*total_block; and
S1.5: sorting the average luminances of all blocks in descending order, so that a metering result is;
$${Y}_{L} = ⌊ \frac{S}{bloc k} {\sum }_{j = 1}^{block} {L}_{j} ⌋ + ⌊ \frac{1 - S}{{total}_{b l o c k} - block - 1} {\sum }_{block + 1}^{total \_ b1ock} {L}_{j} ⌋ ,$$
wherein Lⱼ indicates the average luminance of the selected j-th block, and S is a tolerance parameter which can be set to any value between 0 and 1;
for an N-th frame image in a video stream, input conditions for an auto dimming module are: a current image luminance Y_{N}, a previous-frame image luminance Y_{N-1}, a target image luminance TY, exposure times ET_{N} and ET_{N-1}, analog gain amplification factors GAIN_{N} and GAIN_{N-1}; and a process of determining whether a dimming is required in S2 is as follows:
S2.1: when abs(TY−Y_{N})<range, i.e., the current image luminance is slightly different from the target image luminance, maintaining exposure parameters unchanged, and exiting the auto dimming, wherein abs( ) represents an absolute value function, and range represents a threshold constant; and
S2.2: when abs(TY−Y_{N})>range, entering a dimming process;
in the step S3, following substeps are included:
S3.1: dynamically determining whether to adjust a picture;
S3.2: performing adaptive dark region brightening; and
S3.3: performing dynamical contrast enhancement;
the dynamically determining whether to adjust the picture in step S3.1 specifically comprises following substeps:
S3.11: first, converting the image from an RGB domain to a YUV domain using a formula as follows:
Y=0.1826*R+0.6142*G+0.062*B+16,
U=−0.1006*R−0.3386*G+0.4392*B+128,
V=0.4392*B−0.3989*G−0.0403*B+128;
S3.12: performing histogram statistics on a Y channel, wherein during the histogram statistics, only pixels with a luminance greater than TH_valid are counted, after a luminance histogram Hist_L is obtained, the numbers of pixels across all grayscale levels in Hist_L are summed to obtain Hist_L_sum, then the number of pixels across each grayscale level in Hist_L is divided by Hist_L_sum to obtain a weight for each grayscale level, as expressed in following formula:
Hist_L_wⱼ=Hist_Lⱼ/Hist_L_sum;
S3.13: performing weighted summation on the weight and each grayscale value to obtain a weighted grayscale value of the image, as expressed in following formula:
$$gray\_avg = {\sum }_{j = 0}^{255} Hist\_L {\_w}_{j} * j ,$$
in the luminance histogram Hist_L, the number of pixels with grayscale levels less than TH_GT is counted as left_num and the number of pixels with grayscale levels greater than or equal to TH_GT is counted as right_num, wherein TH_GT defaults to 90, and r_l_ratio is a ratio of a number of pixels on the right side of TH_GT to a number of pixels on the left side of TH_GT, as expressed in following formula:
r_l_ratio=right_num/left_num; and
S3.14: when gray_avg is greater than or equal to TH_GT, considering that the picture is normal and requires no changes;
when r_l_ratio is greater than or equal to 1, considering that the picture is normal and requires no changes; except for the above two cases, considering that the picture is generally dark and requires dynamic brightening;
the adaptive dark region brightening in the step S3.2 specifically comprises following substeps:
S3.21: when a value of gray_avg is small, considering that the picture has a low overall luminance and more enhancement is needed for a dark region, in which case a bimodal distribution may appear such that it is necessary to introduce a ratio r_l_ratio, and the closer the ratio approaches 1, the higher an overall differentiation of the picture, indicating that the luminance/darkness distribution is appropriate, and there is no need to brighten the dark region.
S3.22: designing a curve dynamically adjusted based on gray_avg and r_l_ratio with a formula as follows:
f(x)=(x^{b}*c^{d})(x^{b}*c^{d}+(1−x)^{d}*a^{b}),
wherein x is a grayscale value ranging from 0 to 255, f(x) indicates that an original grayscale value will be mapped to a new grayscale value, wherein a is 0.3, and d is 1;
c is dynamically adjusted based on gray_avg, with a formula as follows:
c_i=min((TH_GT−gray_avg)/(TH_GT−40),1),
c=0.3+c_i*0.3;
b is dynamically adjusted based on r_l_ratio and gray_avg, wherein min represents a smaller one of the two values, with a formula as follows:
$$new\_r \_l \_ratio = \frac{( r\_l {\_ratio}^{3} * 0.3 {5}^{3} )}{( r\_l {\_ratio}^{3} * {0.35}^{3} + {( 1 - r\_l \_ratio )}^{3} * 0 . {5}^{3} )} ,$$
$$b\_i = 1 - c\_i * ( 1 - new\_r \_l \_ratio ) ,$$
$$b = min ( 0.35 + 0.65 * b\_i , 1 ) ;$$
and
S3.23: applying a grayscale value of a new f(x) to the Y channel of the image in the YUV domain to obtain an image YUV_new which is brightened newly;
the dynamic contrast enhancement in the step S3.3 comprises following substeps:
S3.31: first, converting the image YUV_new to an image RGB_new, i.e., converting the image from the YUV domain to the RGB domain;
S3.32: enhancing the RGB_new using an adaptive linear contrast method, with a specific enhancement formula as follows:
$${res\_p}_{m}^{n} = clip ( ( {p}_{m}^{n} - TH\_GT ) * c\_f + TH\_GT , 0 , 255 ) ,$$
wherein, m in
$${p}_{m}^{n}$$
represents a pixel at a certain position, and n represents a certain color channel in RGB, c_f is a contrast enhancement factor, and a formula of c_f is as follows:
c_f=1.4−0.4*b_i,
wherein, a clip operation means that when a calculation result is less than 0, it will be truncated to 0, and when it is greater than 255, it will be truncated to 255, wherein
$${res\_p}_{m}^{n}$$
is a final graphical result.

2. The dynamic luminance calibration (D.L.C.) method according to claim 1, wherein the dimming process in step S2.2 comprises correcting an exposure model and calculating the exposure parameters, and the correcting the exposure model specifically comprises following substeps:
S2.21: according to a working principle of an image sensor, under a condition of unchanged scenes, providing following relationship:
GAIN_{N+1}−GAIN_{N}=K₁*(Y_{N+1}−Y_{N}),
ET_{N+1}−ET_{N}=K₂*(Y_{N+1}−Y_{N});
S2.22: setting K to an initial value which needs to be corrected in time for changing scenes, and in the dimming process, when one of GAIN and ET changes, recalculating a value of K of a current frame based on a variation of the luminance, and when both change at the same time, expressing the value of K as follows:
$${K}_{1} = {K}_{1} ⋆ \sqrt[2]{\frac{{( {Y}_{N + 1} - {Y}_{N} )}^{2}}{( G A I {N}_{N + 1} - G A I {N}_{N} ) * ( E {T}_{N + 1} - E {T}_{N} )}} ,$$
$${K}_{2} = {K}_{2} * \sqrt[2]{\frac{{( {Y}_{N + 1} - {Y}_{N} )}^{2}}{( G A I {N}_{N + 1} - G A I {N}_{N} ) * ( E {T}_{N + 1} - E {T}_{N} )}} ; .$$
S2.23: in order to improve the robustness of auto exposure during endoscope movement, taking the value of K as a weighted value of the current frame and previous frames, and after an estimated value of K is obtained, estimating a variation of the exposure parameters based on a difference between a luminance of the current frame and a target luminance of the current frame.

3. The dynamic luminance calibration (D.L.C.) method according to claim 2, wherein calculating the exposure parameters specifically comprises following substeps:
S2.24: introducing n damping coefficients p to further adjust an intermediate process of dimming while a dimming speed can also be adjusted through the damping coefficients, wherein the damping coefficients p are expressed as follows:
$$p = \{ \begin{matrix}{p}_{1} , & t h \leq t {h}_{1} \\ ⋮ & ⋮ \\ {p}_{i} , & t {h}_{i − 1} < t h \leq t {h}_{i} \\ ⋮ & ⋮ \\ {p}_{n} , & t {h}_{n − 1} < t h \leq t {h}_{n}\end{matrix} ,$$
$$th = ⌊ \frac{abs ( TY − {Y}_{N} )}{{Y}_{N}} ⌋ ; .$$
S2.25: since increasing an exposure time helps improve a signal-to-noise ratio, giving priority to using the exposure time during dimming, and adjusting an analog gain amplification factor only when the exposure time is insufficient to meet a luminance requirement, when GAIN=1, disenabling GAIN, and adjusting ET:
ΔET=└p*K₂*(TY−Y_{N})┘,
ET_{N+1}=ET_{N}+ΔET,
calculating and issuing an actual register value Reg_{ET} to complete the dimming of the current frame.

4. The dynamic luminance calibration (D.L.C.) method according to claim 3, wherein when GAIN≠1, GAIN is adjusted:
ΔGAIN=└p*K₁*(TY−Y_{N})┘,
GAIN_{N+1}=GAIN_{N}+ΔGAIN,
the actual register value Reg_{GAIN} is calculated at this time, and when ΔGAIN is less than a threshold th_g, it is further corrected through D_{gain};
a difference between a magnification factor corresponding to the actual register value and an actual magnification factor of a previous frame is denoted as A, then a relational expression is:
A*abs(D_{gain}−1)=p*K₁*(TY−Y_{N}),
wherein D_{gain}>0, an adjusted D_{gain} can be calculated based on the relational expression, and finally, Reg_{ET} and Reg_{GAIN} are calculated for ending the dimming, and an N+1 frame image is obtained by multiplying by D_{gain} on the N+1 frame image.

5. The dynamic luminance calibration (D.L.C.) method according to claim 1, wherein in step S3.31, a formula for converting the image from the YUV domain to the RGB domain is as follows:
R=1.1644*(Y−16)+1.7928*(V−128),
G=1.1644*(Y−16)−0.2133*(U−127)−0.533*(V−128),
B=1.1644*(Y−16)+2.1124*(U−128).
